(19) United States
(12) Patent Application Publication
(10) Pub. No.: US 2002/0156502 A1
(43) Pub. Date: Oct. 24, 2002

(54) Pressure relief device for helping sleeping

(76) Inventor: Wei Tuan, Taipei (TW)

Correspondence Address: CHARLES E. BAXLEY, 5th Floor, 59 John Street, New York, NY, 10038, US

(21) Appl. No.: 09/839,091

(22) Filed: Apr. 23, 2001

(51) Int. Cl. A61N 1/36

(52) U.S. Cl. 607/2

(57) ABSTRACT

A pressure relief device includes one or more conductive members attached onto the wrist portion of the user for engaging with some of the acupoints of the users and to actuate the acupoints and to relieve the pressures of the users and to facilitate the sleeping of the users. The conductive member may be detachably or resiliently attached to the wrist portion of the user with a clamping device. A casing has a base board attached to the clamping device, for supporting the conductive members, and for supporting control electric parts or elements.

### BACKGROUND OF THE INVENTION

1. Field of the Invention

The present invention relates to a pressure relief device, and more particularly to a pressure relief device for facilitating or for helping sleeping of the users.

2. Description of the Prior Art

More and more pressures and/or stresses and/or burdens have been added to the people today. People may not sleep well if they may not relieve the pressures and/or stresses and/or burdens added or introduced to them. Actually, more and more people may not sleep well today.

The present invention has arisen to mitigate and/or obviate the afore-described disadvantages of the conventional sleeping problems.

### SUMMARY OF THE INVENTION

The primary objective of the present invention is to provide a pressure relief device for suitably relieving the pressure of people and for facilitating or for helping sleeping to people.

In accordance with one aspect of the invention, there is provided a pressure relief device comprising at least one conductive member, means for engaging the conductive member onto a wrist portion of a user and to engage with some of the acupoints of the users, and means for energizing the conductive member to actuate the acupoints and to relieve the pressures of the users and to facilitate the sleeping of the users.

The engaging means includes a clamping device attached to the conductive member for detachably or resiliently attaching the conductive member onto the wrist portion of the user.

The clamping device includes at least one depression formed therein for receiving projections of the wrist portion of the user, such as the carpals, the pisiform or the like of the users, and for allowing the users to comfortably wear the pressure relief device.

The clamping device includes an outwardly extended flap for forming an opening in the clamping device and for allowing the clamping device to be engaged onto the wrist portion of the user.

A casing is further provided and includes a base board attached to the clamping device, for supporting the conductive member.

The base board includes a bottom portion, the conductive member is attached to the bottom portion of the base board for engaging with some of the acupoints of the users.

Another conductive member may further be provided and attached to the clamping device for engaging with the other acupoints of the users.

One or more batteries may further be provided and coupled to the conductive member for energizing the conductive member.

The energizing means includes a micro control unit and a voltage generator coupled to the conductive member for actuating the conductive member.

The energizing means includes a micro control unit and an output control circuit coupled to the conductive member for actuating the conductive member. A device may further be provided for generating vibrations and/or for massaging the users.

Further objectives and advantages of the present invention will become apparent from a careful reading of a detailed description provided hereinbelow, with appropriate reference to accompanying drawings.

### BRIEF DESCRIPTION OF THE DRAWINGS

FIG. 1 is a perspective view of a pressure relief device in accordance with the present invention;

FIGS. 2, 3, 4 are partial exploded views of the pressure relief device, viewing from different angles;

FIG. 5 is a block diagram of the pressure relief device; and

FIG. 6 is an electric circuit of the pressure relief device.

### DETAILED DESCRIPTION OF THE PREFERRED EMBODIMENT

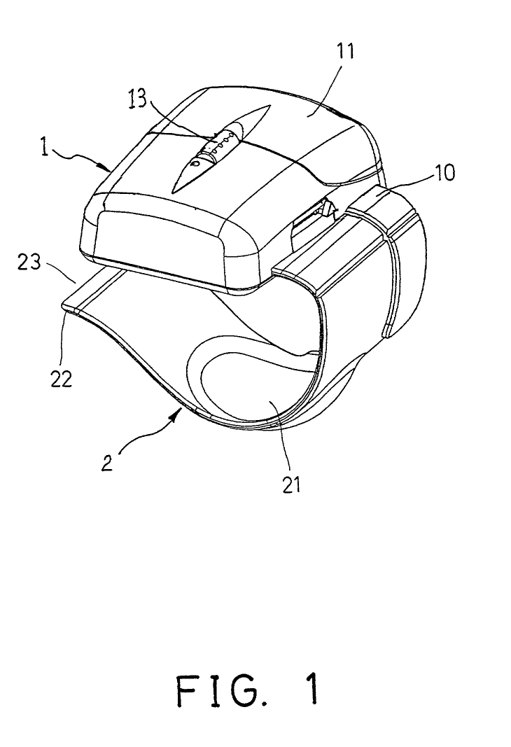

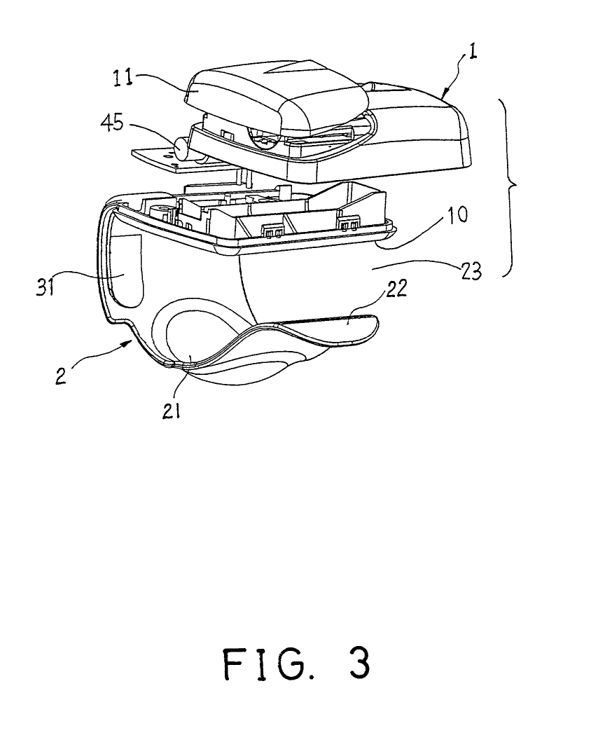

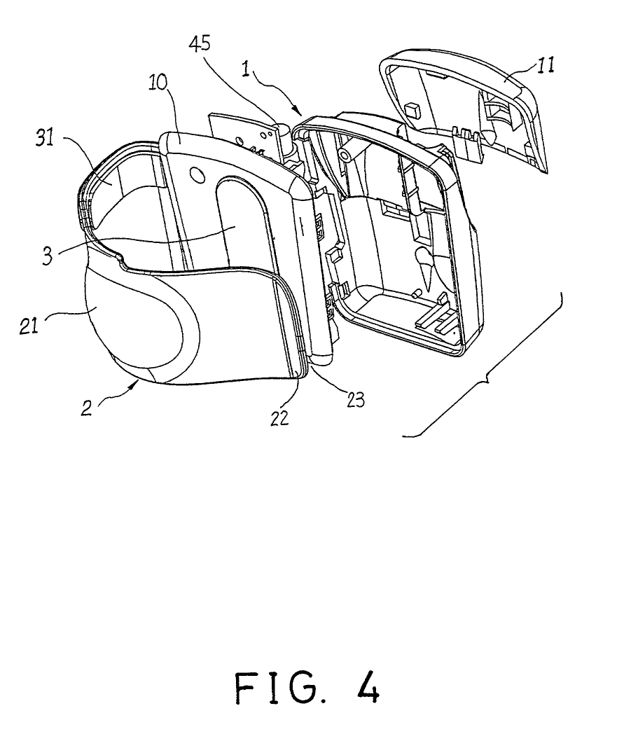

Referring to the drawings, and initially to FIGS. 1-4, a pressure relief device in accordance with the present invention comprises a casing 1 including a base board 10 and an engaging or clamping device 2 attached to the base board 10 or extended from the base board 10 for detachably or resiliently clamping onto the wrist portion of the users. The clamping device 2 is preferably made of resilient materials, such as the plastic or rubber or composite materials for providing a suitable resilience to clamp onto the wrist portion of the users. The clamping device 2 includes one or more depressions 21 formed therein for receiving the projections or the bones of the wrist portion of the user, such as the carpals, the pisiform or the like, and for allowing the users to comfortably wear the pressure relief device. The clamping device 2 includes an outwardly bent or curved or extended flap 22 for forming or defining an opening 23 in the clamping device 2 and for allowing and for facilitating the engagement of the wrist portion of the user into the clamping device 2.

A conductive panel or member 3 is attached onto the bottom portion of the base board 10 for engaging onto some of the acupoints or the acupuncture points in the wrist portion of the user, particularly the Dairyo and the Naikan acupoints. Another conductive panel or member 31 is further provided and attached onto one side portion or an intermediate portion of the clamping device 2 for engaging onto some of the acupoints or the acupuncture points in the wrist portion of the user, particularly the Shinmon acupoint. The clamping device 2 is used for clamping or for engaging the conductive members 3, 31 onto the acupoints or the acupuncture points in the wrist portion of the user. The clamping device 2 thus may also be the engaging strap for engaging onto the wrist portion of the user with a hook and loop securing device, for example.

Two conductors 15, 151 are disposed in the casing 1 and coupled to the conductive members 3, 31, and one or more batteries 12 are engaged and secured between the conductors 15, 151 for energizing the conductive members 3, 31 and/or the other electric elements or parts received in the casing 1. A switch 13 may be provided for switching the electric circuit of the pressure relief device, and is preferably extended outward of the casing 1 for allowing the users to operate or to actuate the switch 13. A cover 11 may be detachably or openably secured to the casing 1 for enclosing the batteries 12 in the casing 1 and for allowing the batteries 12 to be replaced or changed with the other ones.

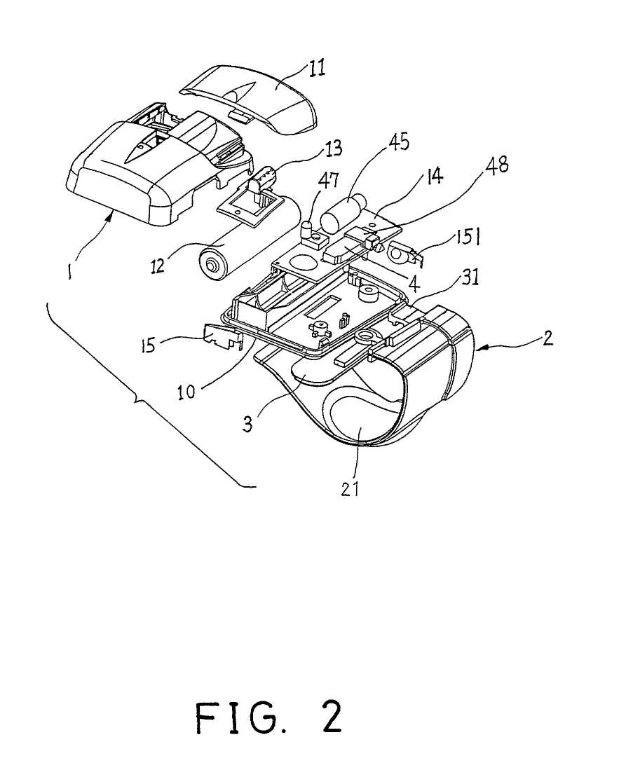

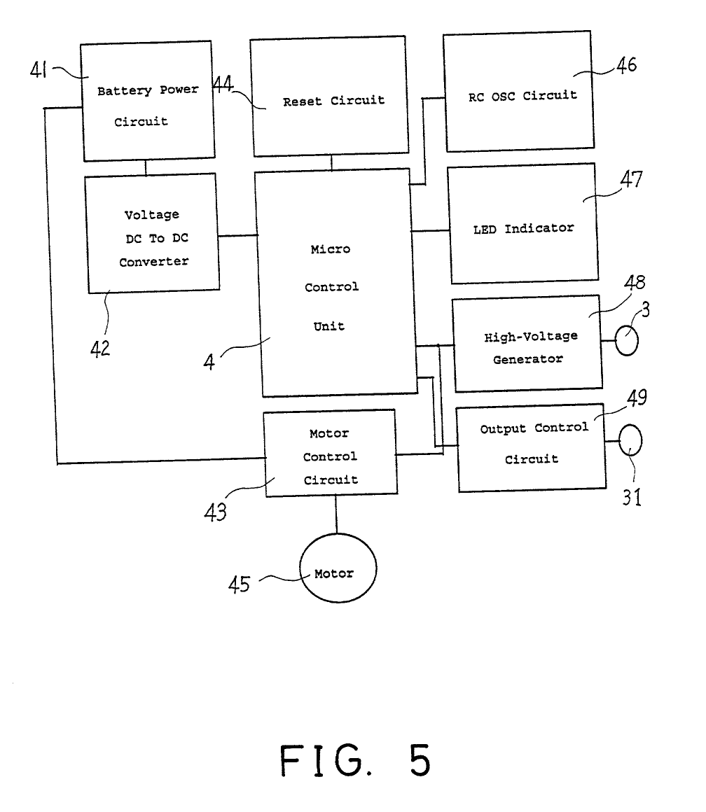

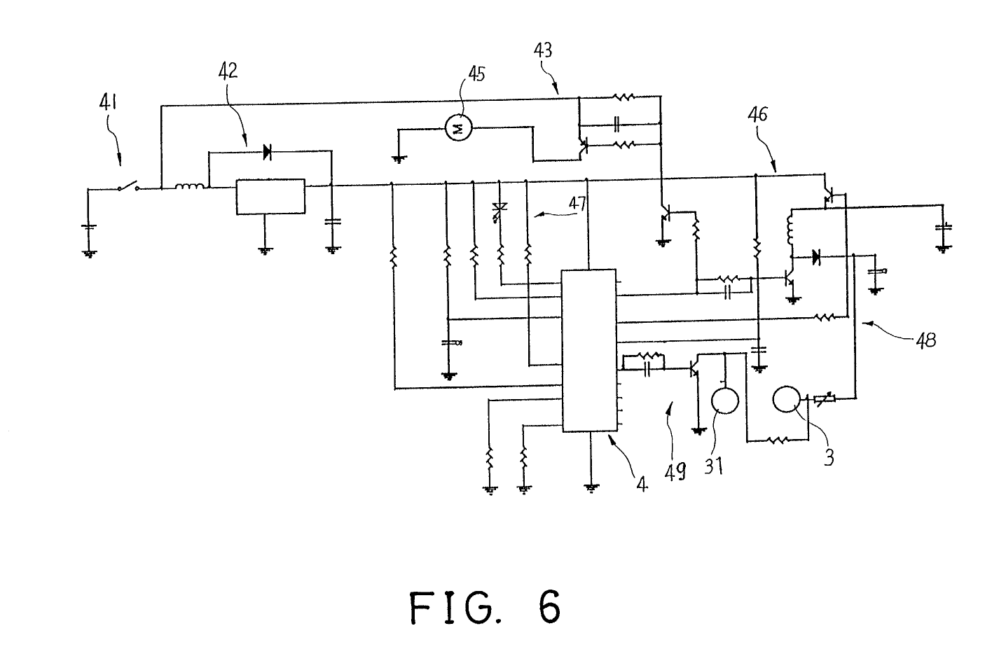

Referring next to FIGS. 5, 6 and again to FIG. 2, the pressure relief device further includes an electric circuit or a control circuit or device to control or to actuate the conductive members 3, 31. The control circuit includes a central processing unit or a micro control unit 4, a battery power circuit 41 or the switch 13 and a voltage converter 42 coupled to the micro control unit 4 and coupled to the batteries 12, for converting the direct current (DC) of the batteries 12 into the required DC power for energizing the electric elements, particularly the micro control unit 4. A motor control circuit 43 is coupled between the battery power circuit 41 or the switch 13 and the micro control unit 4 and is coupled to a motor 45 for actuating or controlling the motor 45. The motor 45 may be used to generate vibrations for massaging the users or to relieve the pressure of the users, for example.

An oscillating circuit 46 may be coupled to the micro control unit 4 and may be used to generate an oscillating signal or the like. One or more light emitting diode indicators 47 may also be provided and coupled to the micro control unit 4 and may be used to generate a warning light signal or the like, for indicating that the pressure relief device is in use. A high voltage generator 48 may be coupled to the micro control unit 4 and the motor control circuit 43 and may be coupled to the conductive member 3 for supplying an electric energy or an electric shock to the conductive member 3, for example. An output control circuit 49 may be coupled to the micro control unit 4 and may be coupled to the other conductive member 31 for supplying an electric energy or an electric shock or electric pulses or the like to the conductive member 31. The electric shock or the electric pulses or the like may be supplied to the conductive member 31 in a predetermined time interval, in a predetermined voltage, in a predetermined wave, and/or in a predetermined frequency, etc., for perioridically stimulating the acupoints of the users, and for relieving the pressures of the users, and thus for facilitating and helping the sleeping of the users. A reset circuit 44 may be coupled to the micro control unit 4 and may be used to reset the electric circuit of the pressure relief device, for example.

Accordingly, the pressure relief device in accordance with the present invention may be used for suitably relieving the pressure of people and for facilitating or for helping sleeping to people.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A pressure relief device comprising:
at least one conductive member,
means for engaging said at least one conductive member onto a wrist portion of a user, and
means for energizing said at least one conductive member.

2. The pressure relief device according to claim 1, wherein said engaging means includes a clamping device attached to said at least one conductive member for attaching said at least one conductive member onto the wrist portion of the user.

3. The pressure relief device according to claim 2, wherein said clamping device includes at least one depression formed therein for receiving projections of the wrist portion of the user.

4. The pressure relief device according to claim 2, wherein said clamping device includes an outwardly extended flap for forming an opening in said clamping device and for allowing said clamping device to be engaged onto the wrist portion of the user.

5. The pressure relief device according to claim 2 further comprising a casing having a base board attached to said clamping device, for supporting said at least one conductive member.

6. The pressure relief device according to claim 5, wherein said base board includes a bottom portion, said at least one conductive member is attached to said bottom portion of said base board.

7. The pressure relief device according to claim 2 further comprising a second conductive member attached to said clamping device.

8. The pressure relief device according to claim 1 further comprising a casing having a base board for supporting said at least one conductive member.

9. The pressure relief device according to claim 8, wherein said energizing means includes at least one battery coupled to said at least one conductive member for energizing said at least one conductive member.

10. The pressure relief device according to claim 8, wherein said energizing means includes a micro control unit and a voltage generator coupled to said at least one conductive member for actuating said at least one conductive member.

11. The pressure relief device according to claim 8, wherein said energizing means includes a micro control unit and an output control circuit coupled to said at least one conductive member for actuating said at least one conductive member.

12. The pressure relief device according to claim 1 further comprising means for generating vibrations.
